(19) United States
(12) Patent Application Publication
(10) Pub. No.: US 2010/0145166 A1
(43) Pub. Date: Jun. 10, 2010

(54) Integrated Instrumentation System and Method for Assessing Feeding Readiness and Competence in Preterm Infants

(76) Inventors: Rita H. Pickler, Richmond, VA (US); Paul A. Wetzel, Richmond, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/632,272

(22) Filed: Dec. 7, 2009

Related U.S. Application Data
(60) Provisional application No. 61/121,306, filed on Dec. 10, 2008.

(51) Int. Cl. A61B 5/00; G06N 5/02

(52) U.S. Cl. 600/301; 706/52

(57) ABSTRACT

An objective, automated approach for the detection and identification of the suck, swallow and breathe parameters, which approach is vital for successful feeding in preterm infants during bottle feeding. An instrumentation system measures and processes of suck, swallow, and breathe waveforms. The analog signals are digitized by analog-to-digital (A/D) converters. A robust method based on correlation techniques and matched filters is used to detect and identify individual occurrences of suck, swallow and breathe. The results of the correlation method results in an occurrence matrix which contains the start time of the detected event (suck, swallow and/or breathe), the correlation coefficient, or probability of certainty and duration of the suspected event. The occurrence matrix is then used as a measure of coordination of suck, swallow and breathe to determine feeding readiness and competence.

### BACKGROUND OF THE INVENTION

1. Field of the Invention

The present invention generally relates to an integrated instrumentation system to measure preterm infant behavior during bottle feeding and a method for assessing feeding readiness and competence in preterm infants, and more particularly to an instrumentation system that allows the measurement of suck, swallow, breathe and cardiac waveforms and processing of the waveforms to provide an objective, automated assessment of feeding readiness and competence in preterm infants.

2. Background Description

Competence at feeding is a criterion for hospital discharge for infants who are born preterm. Once physiological stability has been attained, a major challenge for preterm infants is achievement of oral feeding competence. Sucking activity is often used as an indicator of feeding readiness. Assisting the preterm infant achieve bottle-feeding competence is a primary responsibility of the nursing staff. Despite this responsibility, there continues to be a paucity of information available to support nurses in their decision making regarding preterm infant feeding. Conventional wisdom in many nurseries follows numerous reports that safe, bottle feeding is dependent on suck-swallow-breathe coordination; however, suck-swallow-breathe coordination has not been correlated with actual feeding performance. Moreover, few nurseries have specific policies governing the initiation or progression of bottle-feedings.

### SUMMARY OF THE INVENTION

It is therefore an object of the present invention to provide an objective, automated approach for the detection and identification of the suck, swallow and breathe parameters, which approach is vital for successful feeding in preterm infants during bottle feeding.

This invention allows for easy measurement of the main components of feeding activity: sucking, swallowing, and breathing. According to the invention, there is provided an instrumentation system that allows the measurement and processing of suck, swallow and breathe waveforms. These waveforms are amplified and processed. The analog signals from the integrated instrumentation system are first sampled and digitized by an analog-to-digital (A/D) converter at a rate of 1,000 samples per second. The sampled data from individual channels of suck, swallow and breath are then digitally stored for later analysis. A robust method based on correlation techniques and matched filters is used to detect and identify individual occurrences a suck, swallow and breathe. The correlation method involves methods of up and down sampling to account for differences between waveform duration compared to the representative signal of interest. The results of the correlation method results in an occurrence matrix which contains the start time of the detected event (suck, swallow and/or breathe), the correlation coefficient, or probability of certainty and duration of the suspected event. The occurrence matrix is then used as a measure of coordination of suck, swallow and breathe to determine feeding readiness and competence.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is a block diagram of the instrumentation system according to the invention;

FIG. 2 is a schematic and block diagram showing the details of the suck measurement circuitry;

FIG. 3 is a schematic and block diagram showing the details of the swallow measurement circuitry;

FIG. 4 is a schematic and block diagram showing the details of the breathe measurement circuitry; and

FIG. 5 is a flow diagram of the implementation of the suck, swallow and breath algorithms according to the invention.

### DETAILED DESCRIPTION OF A PREFERRED EMBODIMENT OF THE INVENTION

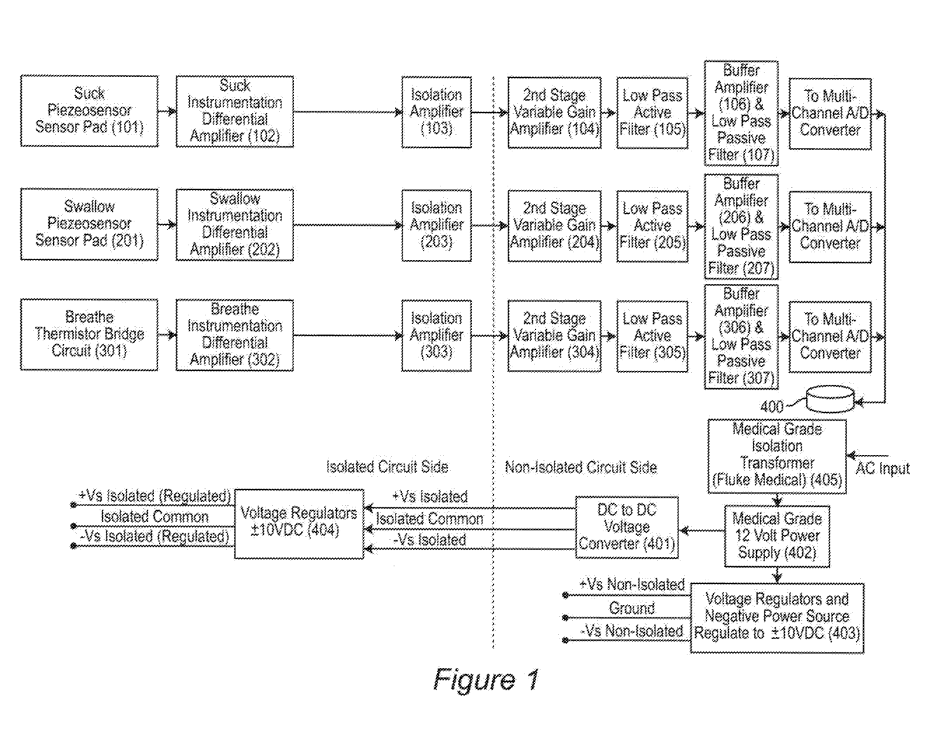

Referring now to the drawings, and more particularly to FIG. 1, there is shown a block diagram of the instrumentation system. As seen in the block diagram, the instrumentation system is composed of individual specialized circuits for the measurement and processing of suck, swallow, and breathe. More particularly, there is provided a piezoelectric sensor pad 101 to detect suck, and another piezoelectric sensor pad 201 to detect swallow. A thermistor bridge circuit 301 is used to detect breathe. To minimize The effects of noise, the front end of each sensor is differentially amplified with fixed but programmable gain. Specifically, a suck instrumentation differential amplifier 102 receives the output of the stick piezoelectric sensor pad 101, a swallow instrumentation differential amplifier 202 receives the output of the swallow piezoelectric sensor pad 201, and a breathe instrumentation differential amplifier 302 receives the output of the breathe thermistor bridge circuit 301. The outputs from the other differential amplifiers 102, 202 and 302 are then applied to individual isolation amplifiers 103, 203, and 303, respectively, which provide electrical isolation from ground. The circuitry described thus far constitutes the isolated circuit side Of the instrumentation system.

The outputs of each isolation amplifier 103, 203, and 303 corresponding respectively to suck, swallow and breathe signals are then applied to an additional amplification stage 104, 204, and 304 followed by individual active low pass filters 105, 205, and 305, respectively. Additional buffer amplifiers and low pass passive filters 106, 206, and 306, respectively, are then used to minimize any additional remnant noise associated with an isolated DC to DC power supply.

A DC to DC power supply converter 401 provides isolated voltages, +Vs, −Vs, and common, to isolated voltage regulators 404 which, in turn, is used to provide power for the first stage differential amplifiers 102, 102, and 302 and the isolation amplifiers 103, 203, and 303. This isolated power source provides a high level of patient safety by isolating all patient connections and references from ground. A single voltage medical grade power supply 402, powered by a medical grade, isolation transformer 405, provides power to the instrumentation system. The voltage from the power supply 402 is applied to the DC to DC power supply converter 401 and, in addition, is applied to voltage regulators 403 which creates a regulated negative voltage. The voltages from voltage regulators 403 are then used to power the non-isolated circuitry.

Analog signals from the instrumentation system are first sampled and digitized by analog-to-digital (A/D) converters 107, 207, and 307 at a rate of 1,000 samples per second. The sampled data from individual channels of suck, swallow, and breathe are then digitally stored in data storage device 400 for later analysis. Any multi-channel analog to digital converter can be used to digitize the required signals of interest. To provide sufficient tune resolution all signals are sampled at a rate of 1,000 samples per second at a minimum resolution of 12 bits or higher. The sampled data are stored on hard drive 400 in 16 bit binary format for later processing and analysis.

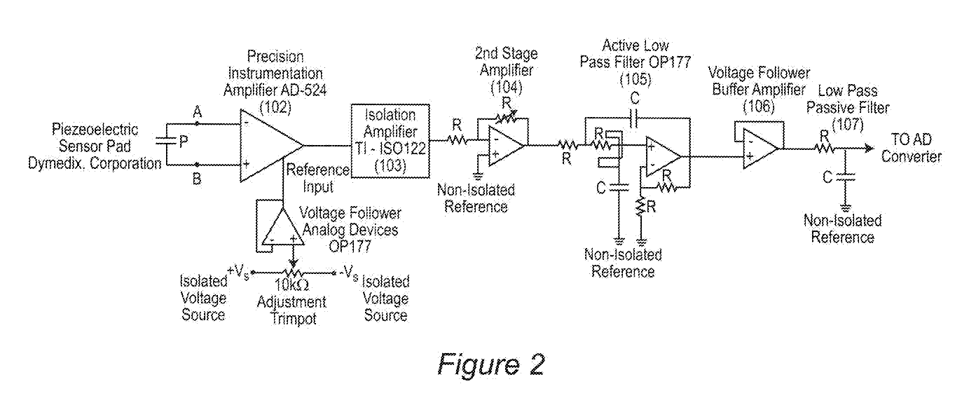

The suck measurement circuit is shown in more detail in FIG. 2. This circuit is comprised of a piezoelectric sensor pad 101, a precision, high instrumentation amplifier 102, an isolation amplifier 103, a second stage inverting amplifier 104, an active low pass active filter 105, a voltage follower buffer circuit 106, and a final stage low pass passive filter 107. In the preferred embodiment, the piezoelectric sensor pad 101 is manufactured by Dymedix Corporation of Shoreview, Minn.; however, other equivalent sensors may be used in the practice of the invention. In the preferred embodiment of the invention, the high gain instrumentation amplifier 102 is a model AD524 amplifier Manufactured by Analog Devices of Norwood, Mass.; however, other equivalent amplifiers may be used in the practice of the invention. In the preferred embodiment of the invention, isolation amplifier 103 is model ISO 124P amplifier manufactured by Texas Instruments of Dallas, Tex.; however, other equivalent amplifiers maybe used in the practice of the invention. In the preferred embodiment of the invention, the operational amplifiers used in second stage inverting amplifier 104, active low pass active filter 105, and buffer amplifier 106 are model OP177 manufactured by Analog Devices; however, other equivalent operational amplifiers may be used in the practice of the invention. After the final stage low pass filter 107, the signal is sampled, digitized and stored for additional signal processing and analysis.

The output leads from a piezoelectric sensor pad are directly connected to the inverting and non-inverting input terminals of the instrumentation amplifier 102 through a two conductor shielded cable which minimizes interference from electrical noise sources. The instrumentation amplifier gain is set to 1,000 via a programmable jumper pin which maximizes the common mode rejection of the instrumentation amplifier. Any offset voltage from the output of the instrumentation amplifier can be minimized by adjustment of the trimpot offset adjustment. The trimpot voltage, is isolated from the instrumentation reference input pin via the voltage follower circuit which again maintains maximum common mode. The output of the instrumentation amplifier is then applied to the of the isolation amplifier 103 which provides electrical and physical isolation between electrical ground and the non-isolated reference. To minimize noise due to the internal isolation amplifier circuit the device is extensively filtered on both the isolated and non isolated sides with inductive chokes and by-pass capacitors per manufacturer's instructions. The output signal from the isolation amplifier 103 is then applied to a second stage inventing amplifier 104 which amplifies the signal further. The amplified signal is then applied to a second order lowspass active Butterworth filter 105 based on a Sallen-Key VCVS (voltage controlled voltage source) design. The filtered signal is then applied to a voltage follower circuit 106 which buffers or isolates the active filter from a final stage passive. RC low pass filter 107 with a break frequency of 150 Hz whose purpose is to remove any noise associated with the isolation amplifier circuitry, power supplies and any other active components prior to data sampling, quantization and digital storage.

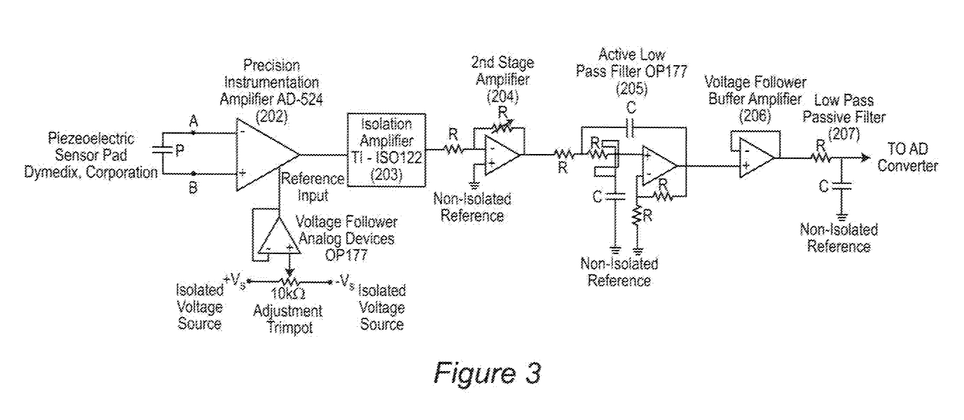

The swallow measurement circuit is shown in more detail in FIG. 3. This circuits comprised of a piezoelectric sensor pad 201, a precision, high gain instrumentation amplifier 202, an isolation amplifier 203, a second stage inverting amplifier 204, an active low pass active filter 205, a voltage follower buffer circuit 206, and a final stage low pass passive filter 207. The components used for this circuit are the same or similar to those used in suck measurement circuit shown in FIG. 2 and described above. After low pass filtering, the signal is sampled, digitized and stored for additional signal processing and analysis.

The output leads front a piezoelectric sensor pad 201 are directly connected to the inverting and non-inverting input terminals of the instrumentation amplifier 202 through a two conductor shielded cable. The instrumentation amplifier gain is set to 1,000 through a programmable jumper which provides maximum common mode rejection of the signal. Any offset voltage from the output of instrumentation amplifier can be minimized by adjustment of the trimpot offset adjustment. The trimpot voltage is isolated from the instrumentation reference input pin via the voltage follower circuit. The output of the instrumentation amplifier to applied to the input of the isolation amplifier 203 which provides electrical and physical isolation between electrical ground and the non-isolated reference. The wire leads to the piezoelectric pad are electrically shielded to minimize noise from electrical sources. To minimize noise due to the internal isolation amplifier circuit, the device is extensively filtered on both the isolated and non isolated sides with inductive chokes and by-pass capacitors per manufacturer's instructions. The output signal from the isolation amplifier 203 is then applied to a second stage inverting amplifier 204 which amplifies the signal further. The amplified signal is then applied to a second order low pass active Butterworth filter 205 based on a Sallen-Key VCVS (voltage controlled voltage source) design. The filtered signal is then applied to a voltage follower circuit 206 which buffers or isolates the active filter from a final stage passive RC low pass filter 207 with a break frequency of 150 Hz whose purpose is to remove any noise associated with the isolation amplifier circuitry, power supplies and any other active components prior to data sampling, quantization and digital storage.

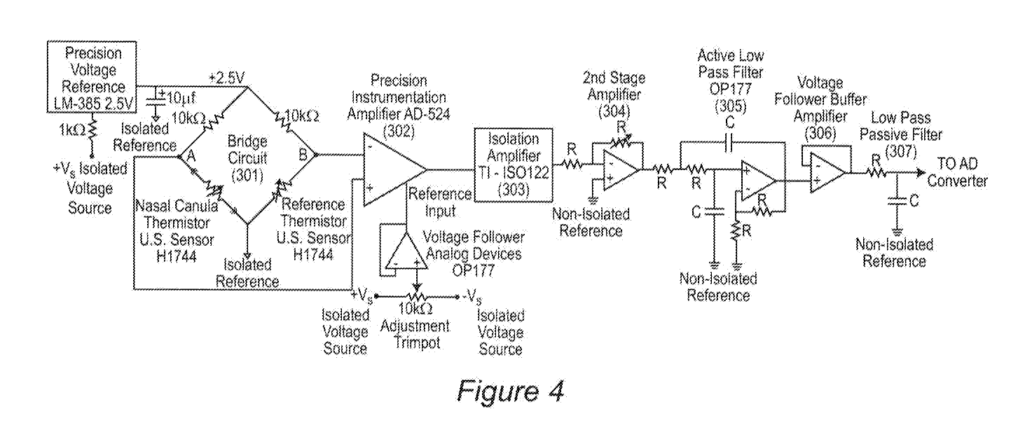

The breathe measurement circuits shown in more detail in FIG. 4. This circuit is composed of a precision voltage reference 300, a bridge circuit 301, differential amplifier 302, isolation amplifier 303, second stage inverting amplifier 304, an active low pass filter 305, buffer amplifier 306, and a passive low pass filter 307. In the preferred embodiment of the invention, the bridge circuit 301 is implemented with fast response acting thermistors model H1744 manufactured by U.S. Sensor of Orange, Calif.; however, other equivalent thermistors may be used in the practice of the invention. The modified pediatric breathing canella which holds the thermistor within the opening of the nose is manufactured by Selzer Medical. The several amplifiers for this circuit are the same or similar to those used in suck measurement circuit shown in FIG. 2 and the swallow measurement circuit shown in FIG. 3 and described above After low pass filtering the signal is sampled, digitized, stored for additional processing and analysis.

The bridge circuit 301 consists of two identical fixed resistors and two identical fast response temperature sensitive thermistors. Together, the combination of a fixed resistor in series with the thermistor forms two separate voltage divider circuits and both sides of the bridge circuit. A precision and highly stable reference voltage 300 is used to excite and provide a fixed voltage source for the bridge circuit. The nasal thermistor is mounted within a modified pediatric nasal canula which holds the thermistor at the opening of the nose. The wire end of the thermistor is connected back to the bridge circuit via a shielded cable and plug. As breathe occurs, the nasal thermistor is both warmed and cooled by air flowing over the thermistor body which produces measurable changes in resistance for all breathe behaviors. To minimize artifact due to changes in ambient temperature and other sources of drift, a second thermistor is incorporated into the opposing branch of the bridge circuit. A differential instrumentation amplifier 302 is used to measure the voltage difference between the A and B points of the bridge with respect to the isolated reference point. The differential measurement across the bridge further minimizes common artifact effects while maximizing changes due to breath alone.

To provide DC offset voltage correction to the instrumentation amplifier 302, a reference voltage adjustment is provided which is buffered thorough the voltage follower and trimpot adjustment circuit. To provide as margin of electrical safety and isolation from ground, the amplified breathe signal is then applied to the isolation amplifier 303 which electrically and physically isolates electrical ground from the isolated reference. To minimize noise due to the internal isolation amplifier circuit, the device is extensively filtered on both the isolated and non-isolated sides with inductive chokes and by-pass capacitors per manufacturer's instructions. The isolation output breathe signal is then applied to a second stage inverting amplifier 304 which amplifies the signal further. The amplified signal is then applied to a second order low pass active Butterworth filter 305 based on a VCVS (voltage controlled voltage source) design. The filtered signal is then applied to a voltage follower circuit 306 which buffers or isolates the active filter from a final stage passive RC low pass filter 307 with a break frequency of 150 Hz whose purpose is to remove any noise associated with the isolation amplifier circuitry, power supplies and any other active components prior to data sampling, quantization and digital storage.

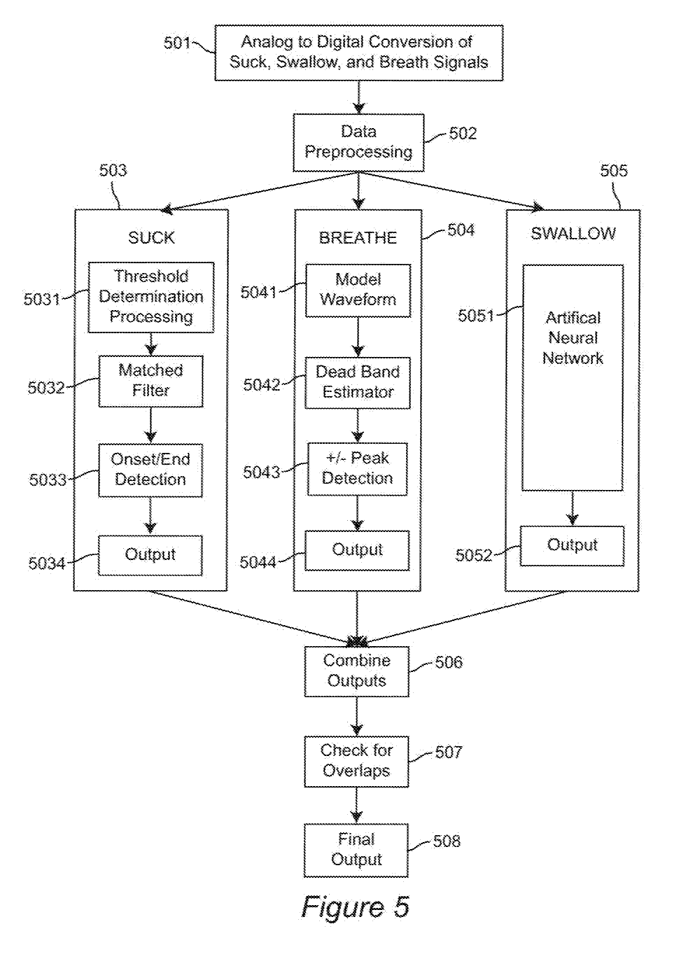

Referring now to FIG. 5, there is shown the automated processing of the suck, swallow and breath signals to provide an objective assessment of feeding readiness and competence in preterm infants. After analog-to-digital conversion 501, the original raw file is saved, and from it the individual suck, swallow, and breathe digitized signals are extracted and stored in a data storage device, such as a computer hard drive. The sampled data is separated and extracted and stored as individual files at 502.

The suck algorithm 503 begins with threshold determination processing 5031. The threshold is calculated by taking the average of all of the data values in the suck signal for the entire duration of the feeding (excluding stop time). The data is then smoothed, velocity is computed, and trend criteria is identified. The output from threshold \determination processing 5031 is input to a match filter 5032. A template of an idealized suck event modeled as a Gaussian waveform is passed over the entire suck signal. Correlation values greater than 0.80 correspond to probable event identification. The output of the match filter 5032 is input to onset/end detection 5033. Previously calculated velocity and trend criteria are used to estimate individual event onsets and ends. The output 5034 is 1×n binary array where binary ones mark each event from onset to end and binary zeros correspond to all other points, and n is the length of the signal.

The breathe algorithm 504 begins with the breath model waveform 5041. The breath waveform is modeled as the sum of two sinusoids and a constant. One sinusoid comprises of the actual nasal airflow signal, the other sinusoid corresponds to the drift of the signal over acquisition time, and the constant represents the DC offset. The first derivative (velocity) of the waveform is calculated and then a low pass filter is applied to remove high frequency noise. Then the second derivative (acceleration) is calculated, which in theory returns the nasal airflow signal in its original state with the baseline drift attenuated to about zero. A moving average filter, or smoothing function, is applied to the waveform. This modeled waveform serves as the input for the following steps. (This is an extension of a method previously described in the literature for the detection of sleep apnea.) The output of the model waveform is input to the dead band estimation 5042. A range of the signal above and below zero is dead banded based on the statistical properties of the data. An initial estimation of the dead band region is based on calculation of half of the first and third quartiles. Then, in +/− peak detection 5043, the positive and negative signal peaks are calculated from the dead banded nasal airflow signal Positive peaks correspond to the onset of inhalation, and negative peaks correspond to the onset of exhalation. The output 5044 is a 2×n binary array where the first row corresponds the onset of inspiration, the second row corresponds to the onset of exhalation, and n is the length of the signal. In both arrays the actual event onsets are marked with ones, and all other points are zeros.

The swallow algorithm 505 incorporates a pre-trained artificial neural network (ANN) 5051 which is used to detect the onsets, ends, and duration of individual swallow events as the digitized data is pulsed through. A neural network (NN) is used to detect the onsets, ends, and duration of individual swallow events in be digitized data. A NN is an advanced artificial intelligence technology that mimics the brain's learning and decision making process. A NN consists of a number of nodes with “neuron” connections between the nodes. Neurons are arranged in a layer, and the different layers of neurons, which are connected to other neurons, form the neural network. The manner in which the neurons are interconnected determines the architecture of the NN. When training a NN, the network learns from the input data and gradually adjusts its neurons to reflect the desired outputs. The NN structure provides flexibility in mapping that allows the NN to perform without knowing the form of the function governing the inputs. In addition, NNs can adapt to changes in input and output. That is, if the inputs change, the elements the NN can be adjusted to continue to map the new inputs to the same output. This feature can be highly advantageous in signal processing and pattern recognition.

NNs are well suited for variety of biosignals processing applications and they are often used for pattern recognition and classification. In addition, NNs have been shown to perform faster and more accurately than conventional methods of signals. NNs also have the ability to solve problems that have no algorithmic solution. Like the human brain, a NN works by computational tasks; outputs of these are fed to subsequent layers of “neurons” until the desired final output is achieved. Each neuron's output is given a particular weight (sometimes referred to as the synaptic weight because of its location between neurons), and the weights combined with the computational functions of the individual neurons all approximate some general function that achieves the final output. One of the primary advantages to using a neural network classification approach to the swallow signal is that neural networks have the ability to adapt their synaptic weights to changes in the data in real time.

Training a network occurs by providing the system with repeated examples of data and teaching the network to approximate the desired function. During training, the synaptic weights change with each example of the data. The application of each example in the training set is called one epoch. The complete training, the neural network cycles through many epochs until the outputs reach a predetermined convergence point. Once the network has finished training, the final function consisting of the computational functions of individual nodes along with their weights is what is used for the classification of the digitized data. This process is best suited for recurrent neural networks. Recurrent neural networks have at least one feedback loop (where the output of one neuron is fed back as the input to a previous layer's neuron) or self-feedback loop (where a neuron's output is fed back to its input). These feedback loops alter the way neural networks learn as well as its performance capabilities. The recurrence inherent to recurrent networks enable them to process sequential inputs, making this the appropriate architecture for times series data as it can explore the temporal relationships between events as a potential factor in weight determination.

NNs are well documented in the literature. See, for example, Antonelo E A, Schrauwen B, Stroobandt D. “Event Detection and localization for small mobile robots using reservoir computing”. Neural Networks: August 2008; 21(6):862-71; Panakkat A, Adeli H. “Neural network-models for earthquake magnitude prediction using multiple seismicity indicators”. International Journal of Neural Systems: February 2007; 17(1): 13-33.; Ubeyli E D. “Recurrent neural networks with composite features for detection of electrocardiographic changes in partial epileptic patients”. Computers in Biology and Medicine: March 2008; 38(3): 401-10.; Namikawa J, Tani J. “A model for learning to segment temporal sequences, utilizing a mixture of RNN experts together with adaptive variance”. Neural Networks: December 2008; 21(10): 1466-75.; and Kulkarni R V, Venayagamoorthy G K. “Generalized neuron: feedforward and recurrent architectures”. Neural Networks: September 2009; 22(7): 1011-7.

The output 5052 is a 1×n binary array where binary ones mark the event (from onset to end), binary zeros correspond to all other points, and n is the length of the signal.

Coordination detection is accomplished by combining the outputs 5034, 5044 and 5052 at 506. All the algorithmic outputs are concatenated into a 4×n binary array where n is the length of the feeding signal. The first row are the suck algorithm's output, rows two and three are output from the breathe algorithm, and row four is the output from the swallow algorithm. The columns are summed across all rows creating a coordination vector in which values greater than one correspond to instances of overlapping events at 507, and their location within the vector corresponds to their temporal location in the course of the feeding. This is marked as the “Final Output” 508.

The waveforms associated with sucking, swallowing, and breathing originate from brain-stern central-pattern generators that have been conceptualized as groups of anatomically overlapping and multifunctional groups of interneurons biased to produce specific motor behaviors. For the preterm infant who is learning to feed orally prior to term neurological maturation or the term infant who is learning to feed orally after a disruption due to illness, the critical issue involves coordinating the independent function of the three mechanisms into a single activity, oral feeding. In order for safe oral feeding, swallowing cannot occur at particular times during either sucking or breathing. For example, if swallowing completely overlays sucking, the infant will sputter and lose the liquid bolus created by the suck. If swallowing occurs during the peak of inspiration, the peak of expiration, or at other times when intraoral pressures are high, the infant will choke. At the same time, breath holding (apnea) in order to swallow, while avoiding choking, is also a sign of immaturity and can lead to oxygen deprivation. A coordinated suck-swallow-breathe pattern for a term infant after the first few days of life is ideally 1:1:1 or 2:2:1. A preterm infant will take longer to reach this level of coordination although the infant can still safely feed orally.

To determine the coordination of the algorithmic outputs from suck, swallow, breathe signals are concatenated into a 4×n binary array where n is the length of the feeding signal. For example, if the feeding lasts for 5 minutes, n=300 seconds or 300 columns. The first row will be the suck algorithm's output in terms of component of the suck (beginning, peak, end), rows two and three will be output from the breathe algorithm again in term of component of breath (inspiration, peak, expiration), and row 4 will be the output from the swallow algorithm (beginning, peak, end). The columns will be summed across all rows creating a coordination vector in which values greater than one correspond to instances of overlapping of critical events, and their location within the vector corresponds to their temporal location in the course of the suck-swallow-breathe activity. A feeding will then be classified by the degree to which ideal suck-swallow-breathe activity is obtained, that is, the percent of time during which 1:1:1 coordination is noted. Feedings will also be classified by suck-swallow coordination as the coordination of these two events is more likely to achieve a 1:1 ratio before breathing becomes coordinated.

This robust method based on correlation techniques and matched filters is used to detect and identify individual occurrences of suck, swallow and breathe. The results of the correlation method results in an occurrence matrix which contains the start time of the detected event (suck, swallow and/or breathe), the correlation coefficient, or probability of certainty and duration of the suspected event. The occurrence matrix is then used as a measure of coordination of suck, swallow and breathe to determine feeding readiness and competence.

While the invention has been described in terms of a single preferred embodiment, those skilled in the art will recognize that the invention can be practiced with modification within the spirit and scope of the appended claims.

Having thus described our invention, what we claim, as new and desire to secure by Letters Patent is as follows:

1. An apparatus for assessing feeding readiness and competence in preterm infants comprising:
a first sensor for connecting to an infant for detecting suck;
a second sensor for connecting to an infant for detecting swallow;
a third sensor for connecting to an infant for detecting breathe;
first amplification, sampling and digitizing means for analog-to-digital conversion of a signal from the first sensor;
second amplification, sampling and digitizing means for analog-to-digital conversion of a signal from the second sensor;
third amplification, sampling and digitizing means for analog-to-digital conversion of a signal from the third sensor; and
digital storage means for saving original raw digital files from the first, second, third, and fourth amplification, sampling and digitizing means, said digital storage means extracting and storing individual digitized suck, swallow, and breath signals, the sampled data being separated and extracted and stored as individual files in the digital storage means.

2. The apparatus for assessing feeding readiness and competence in preterm infants recited in claim 1, further comprising:
detection means connected to receive from the digital storage means digitized outputs of the first, second and third amplification, sampling and digitizing means to detect and identify individual occurrences of suck, swallow and breathe;
correlation means receiving the outputs of the detection means for producing an occurrence matrix containing a start time of a suck, swallow and/or breathe detected event, a correlation coefficient or probability of certainty and duration of the event; and
output means responsive to the occurrence matrix for providing a measure of coordination of suck, swallow and breathe as an indication of feeding readiness and competence.

3. A method of assessing feeding readiness and competence in preterm infants comprising the steps of:
attaching first, second, and third sensors to an infant to detect suck, swallow, and breathe, respectively;
sampling and digitizing outputs of each of said first, second, and third sensors;
saving original raw digital files of the digitized outputs of the first, second, and third sensors in a digital storage device, said step of saving including extracting and storing individual digitized suck, swallow, and breath signals, the sampled data being separated and extracted and stored as individual files; and
using one or more of said digital files or individual files to assess feeding readiness and competence in preterm infants.

4. The method of assessing feeding readiness and competence in preterm infants recited in claim 3, wherein the using step includes the steps of:
reading from the digital storage device digitized outputs of the first, second and third sensors to detect and identifying individual occurrences of suck, swallow and breathe;
producing an occurrence matrix containing a start time of a suck, swallow and/or breathe detected event, a correlation coefficient or probability of certainty and duration of the event; and
using the occurrence matrix to provide a measure of coordination of suck, swallow and breathe as an indication of feeding readiness and competence.
